(19) United States
(12) Patent Application Publication
(10) Pub. No.: US 2013/0148779 A1
(43) Pub. Date: Jun. 13, 2013

(54) RADIATION TOMOGRAPHY APPARATUS

(71) Applicant: SHIMADZU CORPORATION, Kyoto-shi (JP)

(72) Inventors: Daisuke NOTOHARA, Kyoto-shi (JP); Kazuyoshi NISHINO, Kyoto (JP)

(73) Assignee: SHIMADZU CORPORATION, Kyoto-shi (JP)

(21) Appl. No.: 13/749,565

(22) Filed: Jan. 24, 2013

(30) Foreign Application Priority Data
Jul. 6, 2010 (JP) 2010-154048

(51) Int. Cl. A61B 6/02

(52) U.S. Cl. CPC A61B 6/025
USPC 378/22

(57) ABSTRACT

Provided is a radiation tomography apparatus that performs radiography for a series of fluoroscopic images while moving a radiation source and a radiation detecting device synchronously in opposite directions to each other and obtains a tomographic image of a subject based on the fluoroscopic images. The radiation tomography apparatus allows to preview the tomographic image obtained through the radiography immediately after completion of the radiography. A preview image for previewing a sectional image of a subject is generated besides a tomographic image for diagnosis. The preview image can be obtained through simpler computing processing. Consequently, an operator sees once the preview image displayed on a display unit prior to the tomographic image for diagnosis, thereby determining to conduct radiography again quickly.

### BACKGROUND

1. Field of the Invention

This invention relates to a radiation tomography apparatus provided with a radiation source and an FPD. In particular, this invention relates to a radiation tomography apparatus in which a series of fluoroscopic images are taken while the radiation source and the FPD move synchronously in opposite directions to each other to obtain a tomographic image of a subject based on the fluoroscopic images.

2. Description of the Related Art

Medical institutions are equipped with a radiation tomography apparatus 51 for obtaining a tomographic image of a subject M. Such radiation tomography apparatus 51 includes a configuration in which a radiation source 53 that emits radiation and an FPD 54 that detects radiation move synchronously to take a series of fluoroscopic images successively, and then the series of fluoroscopic images are superimposed to obtain a tomographic image (see FIG. 14.) In such radiation tomography apparatus 51, during taking a series of fluoroscopic images, the radiation source 53 and the FPD 54 move along a body-axis direction of the subject so as to approach to each other, thereby having the same position in the body-axis direction. Thereafter, the radiation source 53 and the FPD 54 move along the body-axis direction as to be spaced away from each other. Such radiographic apparatus is described, for example, in Japanese Patent Publication No. 2005-013736.

Description will be given of the foregoing operation of taking the tomographic image with the radiation tomography apparatus 51. Firstly, the radiation source 53 intermittently emits radiation while moving. Specifically, the radiation source 53 moves along the body-axis direction of the subject for every completion of irradiation, and again emits radiation. In this way, 74 fluoroscopic images are obtained, and then they are reconstructed into a tomographic image by filtered back projection. The finished image is a tomographic image having a sectional image appearing therein when the subject is cut along a cut surface.

Complicated computation is needed for generating a tomographic image. For generating the tomographic image, the obtained 74 fluoroscopic images are sent into a console for tomographic-image generation where they are reconstructed into a tomographic image. Moreover, according to the conventional radiation tomography apparatus 51, it takes some time from taking the fluoroscopic images until obtaining the tomographic image.

The radiation tomography apparatus 51 is provided with an operator console for inputting operator's instructions. An operator can perform radiography again via the operator console. Specifically, where the tomography image contains a blurred subject image due to movement of a subject during taking a series of fluoroscopic images, for example, fluoroscopic radiography can be performed once again to generate a tomographic image. Moreover, it is supposed that low doses of radiation upon taking the fluoroscopic images cause a dark tomographic image. At this time, the operator confirms that the tomographic image is underexposure. Then the operator can provide an instruction to the radiation tomography apparatus 51 so as to take 74 fluoroscopic images once again with an increased dose of radiation to obtain a new tomographic image.

The conventional configuration, however, has the following problem. That is, there arises a problem that the conventional radiation tomography apparatus 51 has difficulty in adjusting various conditions of radiography.

Complicated calculation should be performed to the fluoroscopic images for obtaining the tomographic image. Thus, the tomographic image cannot be obtained immediately after the fluoroscopic images are taken. It can be decided only after the tomographic image is generated whether or not the tomographic image contains a blurred subject image. Accordingly, the operator will notice blurs in the tomographic image after a while from radiography of the fluoroscopic images.

In the case of spot radiography in which radiation is applied to a portion of the subject and radiography is conducted to the portion to take the fluoroscopy image thereof simply, the fluoroscopic images of the subject can be obtained immediately after the radiography. Consequently, where the fluoroscopic image contains blurs, the operator can adjust the dose of radiation at that time immediately to conduct again radiography to the subject. In the case of taking the tomographic image, however, it takes longer time from the first radiography until conducting the radiography again.

Where the radiation tomography apparatus 51 generates the tomographic image, 74 fluoroscopic images are obtained in advance. Accordingly, it is possible to display the 74 fluoroscopic images immediately after completion of radiography. On the other hand, although the fluoroscopic images as an intermediate image in tomographic-image generation are visible, it is difficult to expect blur conditions of the subject in a tomographic image to be generated. In other words, blur conditions cannot be recognized unless the tomographic image is generated.

In preparation for such blurs of the subject in the tomographic image, a subject is continuously set in the radiation tomography apparatus 51 for a long time from completion of taking the 74 fluoroscopic images until generation of the tomographic image. This increases a burden on the subject. Moreover, upon generating the tomographic image conventionally, the subject may leave the radiation tomography apparatus 51 after obtaining the fluoroscopic images. In this case, since the subject leaves the radiation tomography apparatus 51 upon generating the tomographic image, radiography for the fluoroscopic images cannot be conducted again. Accordingly, a doctor has to diagnose the subject using the blurred tomographic image. In addition, the doctor cannot determine not only blur conditions of the tomographic image but also underexposure/overexposure of the tomographic image, i.e., an extent of exposure before generating the tomographic image. Also in this case, radiography cannot be conducted again rapidly.

### SUMMARY

This invention has been made regarding the state of the art noted above, and its object is to provide a radiation tomography apparatus that performs radiography for a series of fluoroscopic images while moving a radiation source and a radiation detecting device synchronously in opposite directions to each other and obtains a tomographic image of a subject based on the fluoroscopic images. The radiation tomography apparatus allows to preview the tomographic image obtained through the radiography immediately after completion of the radiography.

The above object is fulfilled, according to one example of this invention, by a radiation tomography apparatus including a radiation source for emitting radiation to a subject; a radiation detecting device for detecting the radiation emitted to the subject; a top board for supporting the subject placed thereon between the radiation source and the radiation detecting device; a moving device for moving the radiation source and the radiation detecting device in a direction of movement along the top board synchronously in opposite directions to each other; a movement control device for controlling the moving device; an image generating device for generating fluoroscopic images in accordance with detection signals outputted from the radiation detecting device; a tomographic-image generating device for generating a tomographic image for diagnosis having a sectional image of the subject in any cut surface appearing therein by reconstructing a series of the fluoroscopic images successively taken while the radiation source and the radiation detecting device are moved; a preview-image generating device for superimposing the series of the fluoroscopic images and for generating a preview image having the sectional image of the subject in a given cut surface appearing therein; and a display device for displaying the preview image prior to displaying the tomographic image for diagnosis.

Another example of this invention discloses a radiation tomography apparatus including a radiation source for emitting cone-shaped radiation to a subject; a radiation detecting device for detecting the radiation emitted to the subject; a top board for supporting the subject placed thereon between the radiation source and the radiation detecting device; a moving device for moving an imaging system, constituted by the radiation source and the radiation detecting device, and the top board relatively to each other; a movement control device for controlling the moving device; an image generation device for generating fluoroscopic images in accordance with detection signals outputted from the radiation detecting device; an image-dividing device for dividing, by a predetermined width in a direction of movement, each of the fluoroscopic images taken while the imaging system is moved relatively to the top board, and generating segments having different incident directions of radiation relative to the radiation detecting device; a long fluoroscopic-image generating device for combining segments generated from the fluoroscopic images having an identical incident direction of radiation one another in the direction of movement in order of radiography to generate a long fluoroscopic image, and for successively generating long fluoroscopic images for segments other than a segment in a fluoroscopic image; a preview-image generating device for generating a preview image having the sectional image of the subject in a given cut surface appearing therein based on the long fluoroscopic images; a tomographic-image generating device for generating a tomographic image by reconstructing two or more generated long fluoroscopic images; and a display device for displaying the preview image prior to displaying the tomographic image for diagnosis.

The examples of this invention generate a tomographic image for diagnosis having a sectional image of a subject in any cut surface appearing therein by reconstructing fluoroscopic images that are taken while a radiation source and a radiation detecting device are moved in a direction of movement along a top board synchronously in opposite directions to each other. It takes some time to generate the tomographic image for diagnosis. Consequently, although blurred sectional image of the subject appears in the tomographic image for diagnosis, radiography cannot be conducted again for the fluoroscopic images until the tomographic image for diagnosis is generated. Then, according to the examples of this invention, a preview image used for previewing the sectional image of the subject is generated besides the tomographic image for diagnosis. The preview image is a sectional image for the subject same as that for the fluoroscopic images for diagnosis, but has a limited cut surface in a particular position. As a result, the preview image can be obtained by simpler computing process. Consequently, the operator sees once the preview image displayed on the display device prior to the tomographic image for diagnosis, thereby determining immediately blurred conditions of the subject image appearing in the tomographic image generated from a series of fluoroscopic images. As a result, reconducting of radiography can be determined quickly.

It is more preferable that the preview-image generating device of the radiation tomography apparatus mentioned above generates the preview image by superimposing the series of the fluoroscopic images having an identical shape without shifting.

The configuration mentioned above is one specific aspect of this invention. The preview image is generated by superimposing a series of fluoroscopic images having an identical shape without shifting. Consequently, the preview image can be obtained by merely superimposing the fluoroscopic images without shifting, which results in simpler computing process for generating the preview image.

It is more preferable that the preview-image generating device of the radiation tomography apparatus mentioned above generates the preview image by superimposing the series of the fluoroscopic images with addition-integration processing.

The configuration mentioned above is one specific aspect of this invention. The preview image is generated by superimposing a series of fluoroscopic images with addition-integration processing, which results in ensured generation of the preview image.

Moreover, it is more preferable that the radiation tomography apparatus mentioned above further includes a high-pass filtering device for extracting components on a high-frequency side in the fluoroscopic image, and that the preview-image generating section generates the preview image by superimposing the fluoroscopic images having the components on the high-frequency side extracted therefrom by the high-pass filtering device.

As noted above, the preview image is generated by superimposing the fluoroscopic images having the components on the high-frequency side extracted therefrom by the high-pass filtering device. Consequently, blurred components appearing in the fluoroscopic images are removed and thereafter the fluoroscopic images are superimposed to generate a preview image. Thereby, the preview image with high visibility can be obtained.

Moreover, it is more preferable that the radiation tomography apparatus mentioned above further includes a high-pass filtering device for extracting components on the high-frequency side in the preview image generated by the preview-image generating device, and that the display device displays the preview image having the components on the high-frequency side extracted therefrom.

As mentioned above, the display device displays the preview image having the components on the high-frequency side extracted therefrom, thereby displaying the preview image with high visibility. In the method of generating the preview image by superimposing the fluoroscopic images having the components on the high-frequency side extracted therefrom, image processing has to be performed to every fluoroscopic image. Accordingly, it takes time and effort for image processing. On the other hand, according to the configuration mentioned above, it is only needed to perform image processing to the preview image. Consequently, the preview image with high visibility can be provided through simpler calculations.

The examples of this invention generate a tomographic image for diagnosis having a sectional image of a subject in any cut surface appearing therein by reconstructing the fluoroscopic images that are taken while a radiation source and a radiation detecting device are move in a direction of movement along a top board synchronously in opposite directions to each other. It takes some time to generate the tomographic image for diagnosis. Consequently, although blurred sectional image of the subject appears in the tomographic image for diagnosis, radiography cannot be conducted again for the fluoroscopic images until the tomographic image for diagnosis is generated. Then, according to the examples of this invention, the preview image used for previewing the sectional image of the subject is generated besides the tomographic images for diagnosis. The preview image is a sectional image for the subject same as that for the tomographic image for diagnosis, but has a limited cut surface in a particular position. As a result, the preview image can be obtained by simpler computing process. Consequently, the operator sees once the preview image displayed on the display device prior to the tomographic image for diagnosis, thereby determining immediately blurred conditions of the subject image appearing in the tomographic image generated from a series of fluoroscopic images. As a result, reconducting of radiography can be determined quickly.

### BRIEF DESCRIPTION OF THE DRAWINGS

FIG. 1 is a functional block diagram illustrating an X-ray apparatus according to one example of this invention.

FIG. 2 is a schematic view explaining a principle of generating a tomographic image according to the example of this invention.

FIGS. 3 and 4 are schematic views each illustrating the X-ray apparatus according to the example of this invention.

FIGS. 5 through 7 are schematic views each illustrating operation of the X-ray apparatus according to the example of this invention.

FIG. 8 is a flow chart illustrating the operation of the X-ray apparatus according to the example of this invention.

FIG. 9 is a functional block diagram illustrating an X-ray apparatus according to another example of this invention.

FIGS. 10 through 12 are schematic views each explaining a principle of generating a tomographic image according to the other example of this invention.

FIG. 13 is a functional block diagram illustrating an X-ray apparatus according to one modification of this invention.

FIG. 14 is a schematic view illustrating a conventional X-ray apparatus.

### DESCRIPTION OF THE PREFERRED EMBODIMENTS

Each example of a radiation tomography apparatus according to this invention will be described hereinafter with reference to the drawings. Herein, X-rays in each example correspond to radiation in this invention. An FPD is the abbreviation of a flat panel detector.

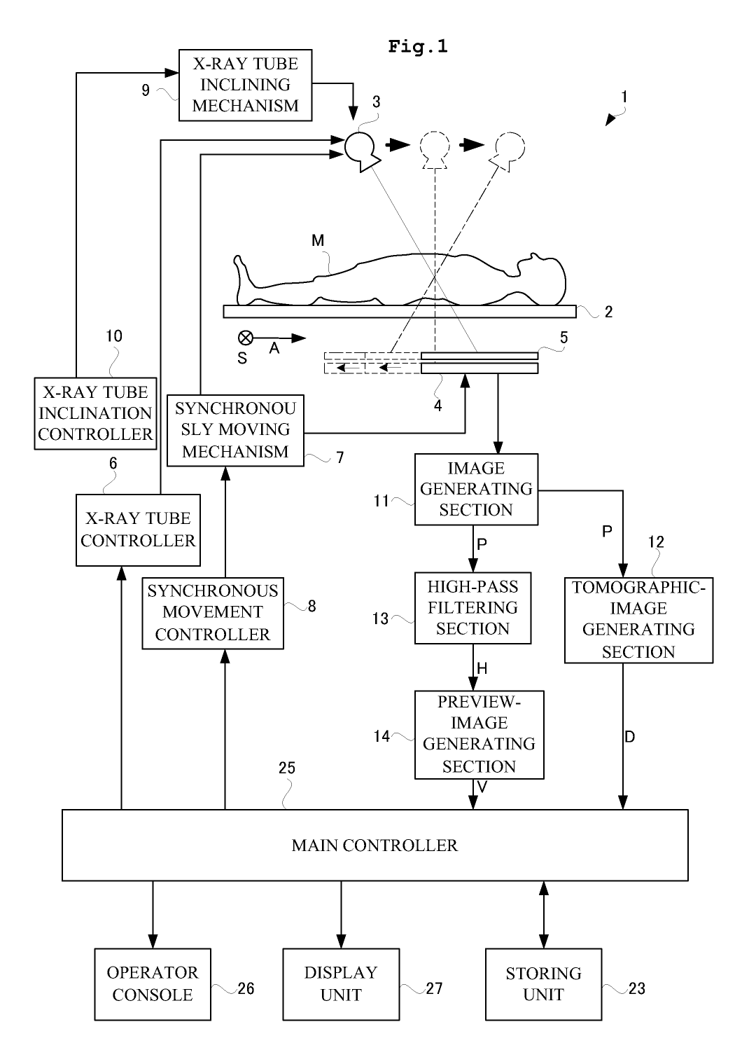

FIG. 1 is a functional block diagram illustrating an X-ray apparatus according to one example of this invention. As shown in FIG. 1, an X-ray apparatus 1 in Example 1 includes a top board 2 for supporting a subject M placed thereon as a target for X-ray tomography; an X-ray tube 3 disposed above the top board 2 (on one face side of the top board 2) for irradiating the subject with a cone-shaped X-ray beam; an FPD 4 below the top board 2 (on the other face side of the top board) for detecting X-rays transmitting through the subject M; a synchronously moving mechanism 7 and a synchronous movement controller 8 for controlling thereof, the synchronously moving mechanism 7 moving the X-ray tube 3 and the FPD 4 synchronously in opposite directions to each other across a site of interest of the subject M while a center axis of the cone-shaped X-ray beam always conforms to a center of the FPD 4; an X-ray grid 5 provided as to cover an X-ray detecting surface of the FPD 4 detecting X-rays for absorbing scattered X-rays. Thus, the top board 2 is placed between the X-ray tube 3 and the FPD 4. The X-ray tube 3 corresponds to the radiation source in this invention. The FPD 4 corresponds to the radiation-detecting device in this invention. Moreover, the synchronously moving mechanism 7 corresponds to the moving device in this invention. The synchronous movement controller 8 corresponds to the movement control device in this invention.

The X-ray tube 3 is constructed to repeat irradiation of the subject M with the cone-shaped and pulsed X-ray beam under control by the X-ray tube controller 6. The X-ray tube 3 has a collimator attached thereto for collimating the X-ray beam into a shape of a pyramid cone. The X-ray tube 3 and FPD 4 constitute imaging system for obtaining X-ray fluoroscopic images.

The X-ray apparatus 1 according to Example 1 further includes a main controller 25 for performing overall controlling en bloc each controller 6, 8, 10, and a display unit 27 for displaying a preview image V and a tomographic image for diagnosis D. The main controller 25 has a CPU, and provides each controller 6, 8, 10 and each section 11, 12, 13, 14, to be mentioned later, by executing various programs. A storing unit 23 stores all data with respect to control of the X-ray apparatus 1 such as parameters used for controlling the X-ray tube 3. The operator inputs each instruction to the X-ray apparatus 1 through an operator console 26. The display unit 27 corresponds to the input device in this invention.

The synchronously moving mechanism 7 moves the X-ray tube 3 and the FPD 4 synchronously. The synchronously moving mechanism 7, under control of the synchronous movement controller 8, moves the X-ray tube 3 linearly along a linear track (in a longitudinal direction of the top board 2) parallel to the body-axis direction A of the subject M. The direction of movement of the X-ray tube 3 and that of the FPD 4 conform to the longitudinal direction of the top board 2. Moreover, the cone-shaped X-ray beam emitted from the X-ray tube 3 during examination is always emitted toward the site of interest of the subject M. The X-ray emission angle is changed, for example, from an initial angle of −20° to a final angle of 20° by changing an angle of the X-ray tube 3. An X-ray tube inclining mechanism 9 performs such changes of the X-ray emission angle. An X-ray tube inclination controller 10 is provided for controlling the X-ray tube inclining mechanism 9.

The synchronously moving mechanism 7 moves the FPD 4, disposed below the top board 2, linearly along the body-axis direction A (the longitudinal direction of the top board 2) of the subject M, synchronously with linear movement of the X-ray tube 3 noted above. The direction of movement thereof is opposite to the direction of movement of the X-ray tube 3. That is, the cone-shaped X-ray beam with various focal points and irradiation directions of the X-ray tube 3 due to movement of the X-ray tube 3 is always received on the entire X-ray detecting surface of the FPD 4. Thus, in one examination, the FPD 4 acquires 74 fluoroscopic X-ray images P, for example, are obtained while the FPD 4 moves synchronously with the X-ray tube 3 in opposite directions. Specifically, the imaging system moves, as opposed to each other, from an initial position shown in solid lines through a position shown in dashed lines toward a position shown in chain lines in FIG. 1. That is, a plurality of X-ray fluoroscopic images are obtained while changing positions of the X-ray tube 3 and the FPD 4. Since the cone-shaped X-ray beam is always received on the entire X-ray detecting surface of the FPD 4, the center axis of the cone-shaped X-ray beam always conforms to the center of the FPD 4 during radiography. During radiography, the FPD 4 moves linearly, and the movement is opposite to the direction of movement of the X-ray tube 3. In other words, the X-ray tube 3 and the FPD 4 may move synchronously in opposite directions to each other along the body axis direction A. The fluoroscopic X-ray image P corresponds to the fluoroscopic image in this invention.

An image generating section 11 is provided downstream of the FPD 4 for generating the fluoroscopic X-ray images P in accordance with detection signals outputted from the FPD 4 (see FIG. 1). Further provided downstream of the image generating section 11 are a tomographic-image generating section 12, a high-pass filtering section 13, and a preview-image generating section 14. The tomographic-image generating section 12 generates a tomographic image D for diagnosis with use of the fluoroscopic X-ray images P. The high-pass filtering section 13 extracts components on a high-frequency side in the fluoroscopic X-ray image P to generate a frequency-processed image H. The preview-image generating section 14 generates a preview image V with use of the frequency-processed image H. The image generating section 11 corresponds to the image generating device in this invention. The tomographic-image generating section 12 corresponds to the tomographic-image generating device in this invention. Moreover, the high-pass filtering section 13 corresponds to the high-pass filtering device in this invention. The preview image generating section 14 corresponds to the preview image generating device in this invention.

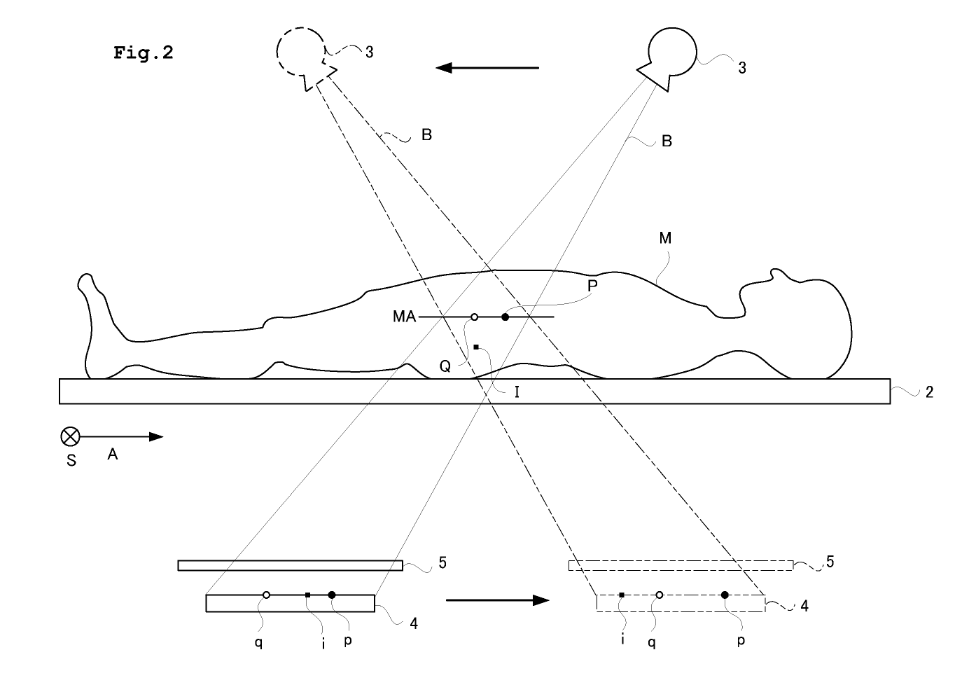

Next, description will be given of the principle of containing a tomographic image with the tomography X-ray apparatus 1 according to Example 1. FIG. 2 is a view illustrating a method of obtaining a tomographic image with the X-ray apparatus according to Example 1. To describe the principle taking a reference cut surface MA parallel to the top board 2 (horizontal relative to the vertical direction) as shown in FIG. 2, for example, the fluoroscopic image generation section 11 generates a series of fluoroscopic X-ray images P while the FPD 4 moves synchronously with and in an opposite direction to the X-ray tube 3 according to an irradiation direction of the cone-shaped X-ray beam B such that points P and Q located in the reference cut surface MA may always be projected to respective fixed points p and q on the X-ray detecting surface of the FPD 4. The profile view of the subject M appears in a series of fluoroscopic X-ray images P while varying in position. Then, the tomographic-image generating section 12 reconstructs a series of fluoroscopic X-ray images P, thereby accumulating images located in the reference cut surface MA (e.g. the fixed points p and q), and resulting in a tomographic X-ray image. On the other hand, a point I not located in the reference cut surface MA appears in the series of subject images as points i, while varying in projected position in the FPD 4. As distinct from the fixed points p and q, such points i become blurred, instead of forming an image, at the step of superimposing the fluoroscopic X-ray images by the tomographic-image generating section 12. An X-ray sectional image showing only the images located in the reference cut surface MA of the subject M is obtained by superimposing a series of fluoroscopic X-ray images P in this way. Thus, when the fluoroscopic X-ray images P are simply superimposed, an X-ray tomographic image at the reference cut surface MA may be obtained. Here, a position of the reference cut surface MA in the vertical direction corresponds to the reference cut surface in this invention.

Further, a similar tomographic image can be obtained from any selected cut surface parallel to the reference cut surface MA, by changing settings of the tomographic-image generating section 12. Although the projected position of the point i described above moves on the FPD 4 during imaging, a speed of this movement increases as a distance increases between the point I before projection and the reference cut surface MA. The series of subject images obtained in this way is reconstructed while shifting in the body-axis direction A at given pitches, whereby a tomographic image for diagnosis D in the cut surface parallel to the reference cut surface MA may be obtained. The tomographic-image generating section 12 reconstructs the series of subject images in this way.

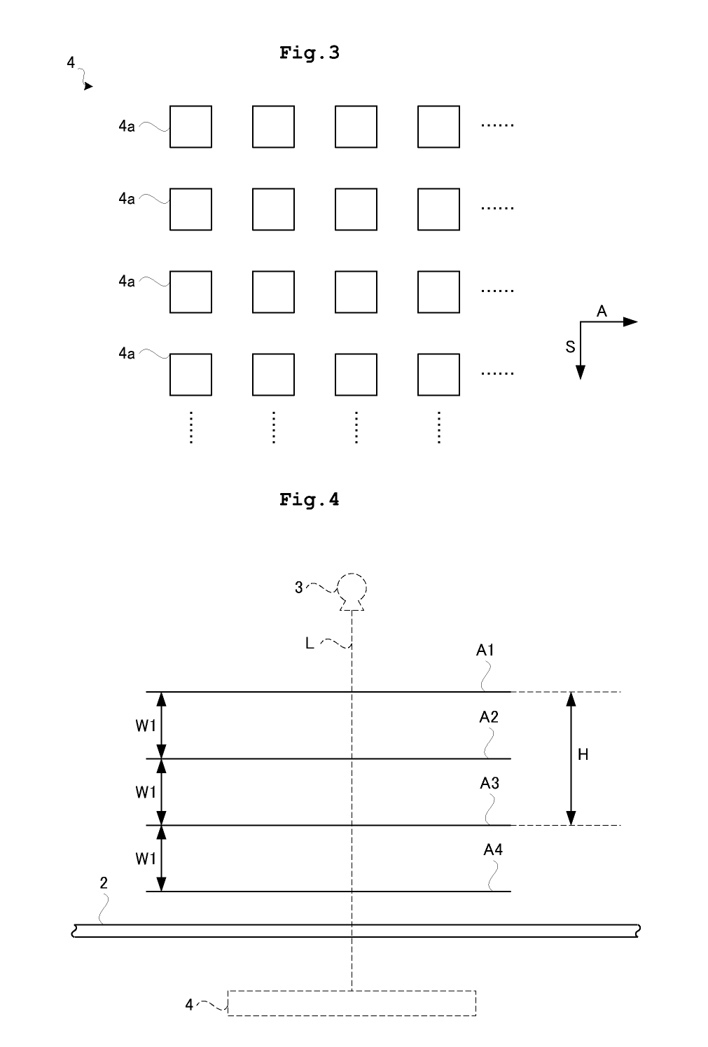

Description will be given of a configuration of the FPD 4. As shown in FIG. 3, the FPD 4 has a detecting surface where radiation is detected with radiation detecting elements 4a arranged longitudinally and transversely. For instance, 1,024 X-ray detecting elements 4a are arranged in the body-axis direction A of the subject M, and 1,024 X-ray detecting elements 4a are arranged in a body-side direction S of the subject M. The arrangement pitch of X-ray detecting elements 4a is 300 μm both in the body-axis direction S and the body-side direction S. Here, the fluoroscopic X-ray image P outputted from the image generating section 11, to be mentioned later, is generated based on detection signals outputted from each of the X-ray detecting elements 4a, and thus pixels are arranged two-dimensionally like the X-ray detecting elements 4a.

Description will be given of the tomographic image D for diagnosis generated by the tomographic-image generating section 12. The tomographic image D for diagnosis contains a sectional image of the subject M cut along any cut surface. This plane is parallel to the top board 2. The tomographic-image generating section 12 generates two or more tomographic images D for diagnosis from a series of fluoroscopic X-ray images P. FIG. 4 illustrates cut surface A1 to A4 for each tomographic image D for diagnosis generated by the tomographic-image generating section 12. Each of the cut surfaces A1 to A4 is orthogonal to a line segment L which connects the X-ray tube 3 to the FPD 4 when the X-ray tube 3 is perpendicularly above the FPD 4. Consequently, the line L is also orthogonal to the top board 2. The cut surfaces A1 to A4 are spaced away from one another by a clearance W1 in a direction where the line L extends. Specifically, the clearance W1 is of approximately 5 mm. FIG. 4 illustrates only four cut surfaces, but in actual forty-one cut surfaces are provided. Consequently, each of the cut surfaces is arranged within a range having a width of 20 cm in a vertical direction.

Description will be given of operation of generating the tomographic image D for diagnosis by the tomographic-image generating section 12. The tomographic-image generating section 12 generates tomographic images D for diagnosis in order from a cut surface nearest to the X-ray tube 3 or a cut surface nearest to the FPD 4. Accordingly, the tomographic image D for diagnosis in the reference cut surface MA cannot be generated immediately.

Next, description will be given of an image appearing in the tomographic image D for diagnosis. An image of the cut surface A2 in FIG. 4 appearing in the cut surface A2 is an image of the subject M cut by the cut surface A2. In actual, however, the tomographic image D for diagnosis contains images of the subject M nearer to the X-ray tube 3 and the FPD 4 than the cut surface A2. In other words, the cut surface A2 contains images of the subject M within a range denoted by H in FIG. 4. A portion in the tomographic image D for diagnosis that contains components the most that are derived from of the cut surface A2. Mentioned above, the tomographic image D contains also components that are derived from other cut surfaces. However, spaced away farther from the cut surface A2 toward the X-ray tube 3 or the FPD 4, contains reduced components of the subject M projected on the tomographic image D for diagnosis. Other cut surfaces in the tomographic image D for diagnosis are similar to this. Consequently, strictly speaking, the tomographic image D for diagnosis contains not only the image itself when the subject M is cut in each cut surface but also images near the cut surfaces. For expediency of explanation, it is assumed that in FIG. 2 the tomographic image D for diagnosis is an image when the subject M is cut at a certain cut surface. Here, the method of obtaining the tomographic image D for diagnosis in this way is called filtered back projection, and computing process conducted by the tomographic-image generating section 12 is complicated.

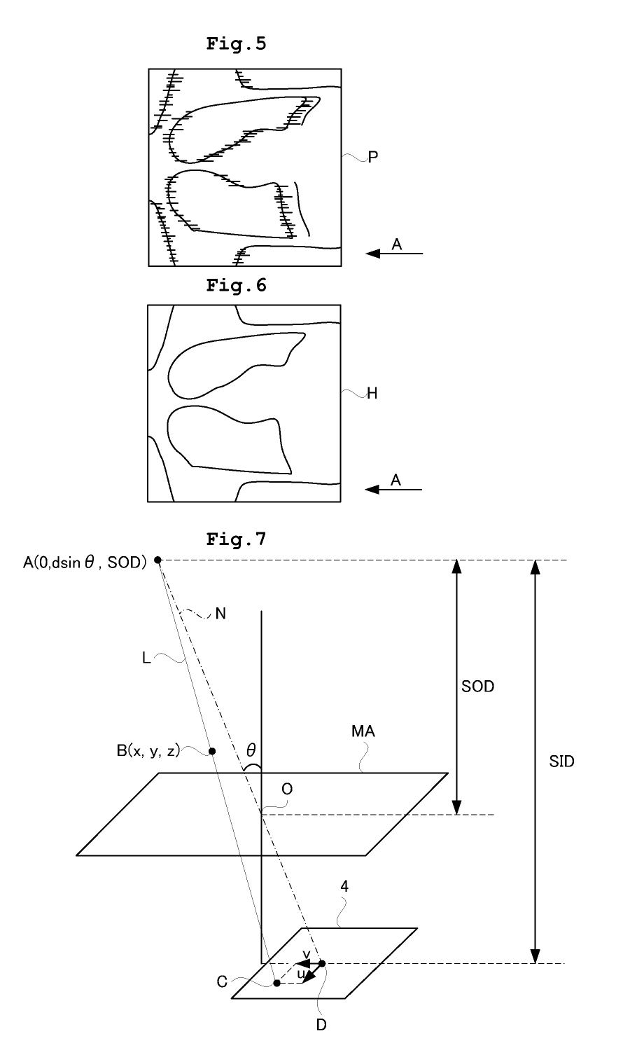

The high-pass filtering portion 13 receives a series of fluoroscopic X-ray images P from the image generating section 11, and generates a frequency-processed image H having components on the high-frequency side in the fluoroscopic X-ray image P extracted therefrom. The high-frequency components are extracted through action of a given matrix filter on pixels forming the fluoroscopic X-ray images P. Since the fluoroscopic X-ray image P is taken while the X-ray tube 3 and the FPD 4 are moved, each of the fluoroscopic X-ray images P is blurred in the direction of movement of the X-ray tube 3 and the FPD 4 (i.e., the body-axis direction A), as illustrated in FIG. 5. The high-pass filtering portion 13 removes low-frequency components, leading to blurs, in the fluoroscopic X-ray images P to generate the frequency-processed image H. As illustrated in FIG. 6, the frequency-processed image H has no blur.

The preview-image generating section 14 receives the frequency-processed images H generated successively from the high-pass filtering portion 13, and superimposes the frequency-processed images H by addition integration processing to generate a preview image V. As described with FIG. 2, when the fluoroscopic X-ray images P (strictly, frequency-processed images H having enhanced visibility than the images) are simply superimposed, a tomographic image on the reference cut surface MA can be obtained. Consequently, the sectional image of the subject M cut by reference cut surface MA appears in the preview image V generated by the preview-image generating section 14.

The preview-image generating section 14 superimposes the frequency-processed images H without shifting in neither the body-axis direction A nor the body-side direction S of the subject M. In this way, as described with FIG. 2, the image of the subject M in positions other than the reference cut surface MA is positively blurred and thus the sectional image of the subject M can be obtained. That is, the preview-image generating section 14 superimposes the fluoroscopic X-ray images P (strictly speaking, frequency-processed images H having enhanced visibility than the images) having the identical shape so as to cover them together completely, thereby superimposing a series of fluoroscopic X-ray images without shifting to generate the preview image V.

The tomographic image D for diagnosis generated by the tomographic-image generating section 12 is more excellent than the preview image V in diagnosis. That is because the tomographic image D for diagnosis contains not only an image itself when the subject M is cut at each cut surface but also the image adjacent to the cut surfaces, which differs from the preview image V. In addition, since the preview image V is always a sectional image in the reference cut surface MA, only one cut surface of the subject M can be diagnosed upon diagnosis for the preview image V. On the other hand, obtaining the preview image V needs no complicated calculation than that upon generating the tomographic image D for diagnosis by the tomographic-image generating section 12, and thus, can be performed immediately after radiography of a series of fluoroscopic X-ray images P.

Moreover, the operator sees once the preview image V even prior to obtaining the fluoroscopic images D for diagnosis, thereby determining immediately blur conditions of the image during fluoroscopic X-ray radiography. That is, when the sectional image of the subject M is blurred in the preview image V, the subject image to be generated in the tomographic image D for diagnosis is similarly blurred. Since the preview image V is also a tomographic image, it clearly indicates blur conditions of the subject image in the tomographic image D for diagnosis generated by the tomographic-image generating section 12. One reason of the blurring of images is movement of subject M on the top board 2 while photographing fluoroscopic X-ray images.

Where the sectional image of the subject M appears dark in the preview image V, the tomographic image D for diagnosis to be generated is similarly dark. This case means that a series of fluoroscopic X-ray images P is obtained in an underexposure state where a dose of X-rays is insufficient. Since the preview image V is also a tomographic image, it clearly indicates an exposure state of the tomographic image D for diagnosis generated by the tomographic-image generating section 12.

Description will be given next geometrically of projecting the tomographic image of the subject M in the preview image V. FIG. 7 is a perspective view illustrating a position of an X-ray focal point (focusing position A), a position of a structure within the subject (a structure position B), and a position where the structure is projected on the FPD 4 (a projecting position C) when a fluoroscopic X-ray image is taken. Here, a clearance in the vertical direction (a direction orthogonal to the top board 2) between the FPD 4 and the focusing position A is denoted by SID. An origin point O is a point where the central axis N of the X-ray beam applied from the X-ray tube 3 always passes irrespective of the position of the X-ray tube 3 during radiography of a series of fluoroscopic X-ray images P. A plane including the origin point O parallel to the top board 2 is a reference cut surface MA. Here, an angle formed by a line connecting the focusing position A to the origin point O and a normal line of the reference cut surface MA is denoted by θ. A clearance in the vertical direction (a direction orthogonal to the top board 2) between the origin point O and the focusing position A is denoted by SOD. The distance d and the angle θ vary in a series of fluoroscopic X-ray images P, whereas the clearance SID and SOD do not vary.

Letting the body-side direction of the subject M be an x-direction, the body-axis direction A of the subject M be a y-direction, and the vertical direction be a z-direction, a coordinate of the focusing position A can be expressed by (0, SOD·tan θ, SOD). That is because the X-ray tube 3 is not moved in the x-direction. Moreover, let a coordinates of the structure position B be expressed by (x, y, z). A line L that passes the points A and B in FIG. 7 can be expressed by

(X,Y,Z)=t(x,y,z)+(1−t)(0,SOD·tan θ,SOD)

Accordingly, there exists a relationship as below between Z and t.

Z=t·z+(1−t)SOD (1)

A position in the vertical direction of the projecting position C is expressed by −(SID−SOD). Accordingly, −(SID−SOD) can be substituted into Z in Equation 1. That is, a relationship of −(SID−SOD)=t·z+(1−t)SOD holds in the projecting position C. This is solved on t to give the following equation:

t=SID/(SOD−z) (2)

The FPD 4 is moved as the X-ray tube 3 is moved. Specifically, the FPD 4 is moved such that central axis N of the X-ray beam always passes the center D of the FPD 4 irrespective of movement of the X-ray tube 3. A position of the central point D can be expressed by

(0,−tan θ(SID−SOD),−(SID−SOD)).

Supposing the projecting position C is apart from the center D by u in the x-direction and by v in the y-direction, a coordinate of the projecting position C can be expressed by

(u,v−tan θ(SID−SOD),−(SID−SOD)).

The projecting position C should pass the line L. Consequently, the left-hand side in the equation of the line L can be replaced with the coordinate of the projecting position C to give the equation below.

(u,v−tan θ(SID−SOD),−(SID−SOD))=t(x,y,z)+(1−t)(0,SOD·tan θ,SOD) (3)

The components in the y-direction in Equation 3 are extracted to give a relationship of v−tan θ(SID−SOD)=t·y+(1−t)SOD·tan θ. This is solved on v to give the equation below:

v=SID·tan θ+t(y−SOD·tan θ) (4)

Equations 2 and 4 derive a relationship below:

v=SID·(y−z·tan θ)/(SOD−z) (5)

That is, Equation 5 reveals that v is in the same position independently of the angle θ when z=0. Here, v is a position of the subject M in the body-axis direction A under assumption that the center D of the FPD 4 is an origin point. In other words, the image of the subject M on the reference cut surface MA where z=0 appears in the same position of a series of fluoroscopic X-ray images P. Accordingly, a series of fluoroscopic X-ray images P are superimposed without shifting to obtain the sectional image of the subject M in the reference cut surface MA.

<Operation of X-Ray Apparatus>

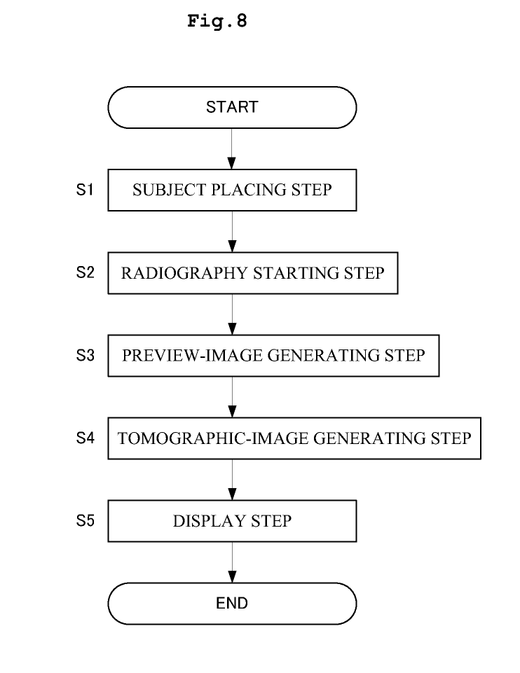

Next, description will be given of operations of the X-ray apparatus 1. As illustrated in FIG. 8, for taking an image of a subject M with use of the X-ray apparatus 1 according to Example 1, firstly the subject M is placed on the top board 2 (Placing step S1.) Thereafter, radiography for a fluoroscopic X-ray image P is started (Radiography Starting Step S2.) Then a preview image V is generated based on a series of fluoroscopic X-ray images P taken (Preview-Image Generating Step S3), and a tomographic image D for diagnosis is generated based on the a series of fluoroscopic X-ray images P (Tomographic-Image Generating Step S4). Finally, the preview image V and the tomographic image D for diagnosis are displayed on a display unit 27 (Display Step S5). Each of these steps will be described hereinafter in order.

<Placing Step S1, Radiography Starting Step S2>

An operator places the subject M on the top board 2, and then provides instructions of starting radiography via an operator console 26 to the X-ray apparatus 1. Then an X-ray tube controller 6 reads out a set value with respect to control of X-ray tube 3, such as tube voltage, tube current, pulse width, etc., that is stored in a storing unit 23. The X-ray tube controller 6 controls the X-ray tube 3 as the set value such that the X-ray tube 3 generates X-rays. X-rays transmitting through the subject M are detected with the FPD 4, and detection signals at this time are sent out to an image generating section 11. The image generating section 11 generates fluoroscopic X-ray images P having a fluoroscopic image of the subject M appearing therein based on the detection signals.

The fluoroscopic X-ray images P are generated in this way for several times while the X-ray tube 3 and the FPD 4 are moved. Consequently, each of the taken fluoroscopic X-ray images P contains the tomographic image of the subject M in various radiography directions. A synchronously moving mechanism 7 moves the X-ray tube 3 and the FPD 4. During radiography, an inclination angle of the X-ray tube 3 is modified as required such that the X-ray tube 3 faces the FPD 4. An X-ray tube inclining mechanism 9 modifies the inclination angle the X-ray tube 3. In this way, 74 fluoroscopic X-ray images P are generated by once radiography.

<Preview-Image Generating Step S3>

A series of fluoroscopic X-ray images are sent out to a high-pass filtering section 13, where frequency processing is performed and they are converted into frequency-processed images H. The frequency-processed images H are sent to a preview-image generating section 14, where they are converted into preview images V having the sectional images of the subject M appearing therein.

<Tomographic-Image for Diagnosis Generating Step S4>

A series of fluoroscopic X-ray images P are also sent to a tomographic-image generating section 12. The tomographic-image generating section 12 starts generation of the tomographic images D for diagnosis by filtered back projection based on a series of fluoroscopic X-ray images P. Two or more tomographic images D for diagnosis are generated while the cut surface of the subject M is changed.

<Display Step S5>

The preview image V is sent out to a display unit 27 prior to completion of generating a first tomographic image D for diagnosis by the tomographic-image generating section 12. Thereafter, the display unit 27 displays the preview image V prior to displaying the tomographic image D for diagnosis. An operator sees once the preview image V, thereby enabling to determine whether or not blur conditions of the subject M and exposure of X-rays during radiography is suitable prior to generation of the first tomographic image D for diagnosis.

Where the operator determines that blur conditions of the subject M and exposure of X-rays are not suitable during radiography, parameters with respect to control of the X-ray tube 3 are adjusted via the operator console 26. Consequently, the process can return again to radiography of the fluoroscopic X-ray image P (Step S2.) Here, it is no need for the operator to wait for generation of the tomographic image D for diagnosis by the tomographic-image generating section 12.

As noted above, Example 1 has a configuration of generating a tomographic image D for diagnosis having a sectional image of the subject M in any cut surface appearing therein by reconstructing fluoroscopic X-ray images P that are taken while the X-ray tube 3 and the FPD 4 are moved in a direction of movement along the top board 2 (the body-axis direction A of the subject M) synchronously in opposite directions to each other. It takes some time to generate the fluoroscopic images D for diagnosis. Consequently, although blurred sectional image of the subject M appears in the fluoroscopic images D for diagnosis, radiography cannot be conducted again for the fluoroscopic X-ray images P until the tomographic images D for diagnosis are generated. Then, according to Example 1, the preview images V used for previewing the sectional image of the subject M are generated besides the tomographic images D for diagnosis. The preview image V is a sectional image of the subject M like the fluoroscopic images D for diagnosis, but has a limited cut surface in a particular position. As a result, the preview image V can be obtained by simpler computing process. Consequently, the operator sees once the preview image V displayed on the display unit 27 prior to the fluoroscopic images D for diagnosis, thereby determining immediately blur conditions of the image of the subject M appearing in the tomographic image D for diagnosis generated from a series of fluoroscopic X-ray images P. As a result, reconducting of radiography can be determined quickly.

The preview image V is generated by superimposing a series of fluoroscopic X-ray images P having an equal shape without shifting. Consequently, the preview image V can be obtained by simply superimposing the fluoroscopic images together without shifting, which results in simpler computing process for generating the preview image V.

As noted above, the preview image V is generated by superimposing the fluoroscopic X-ray images P having the components on the high-frequency side extracted therefrom. Consequently, blurred components appearing in the fluoroscopic X-ray images P are removed and thereafter the fluoroscopic X-ray images P are superimposed to generate the preview image V. Thereby, the preview image V with high visibility can be obtained.

### Example 2

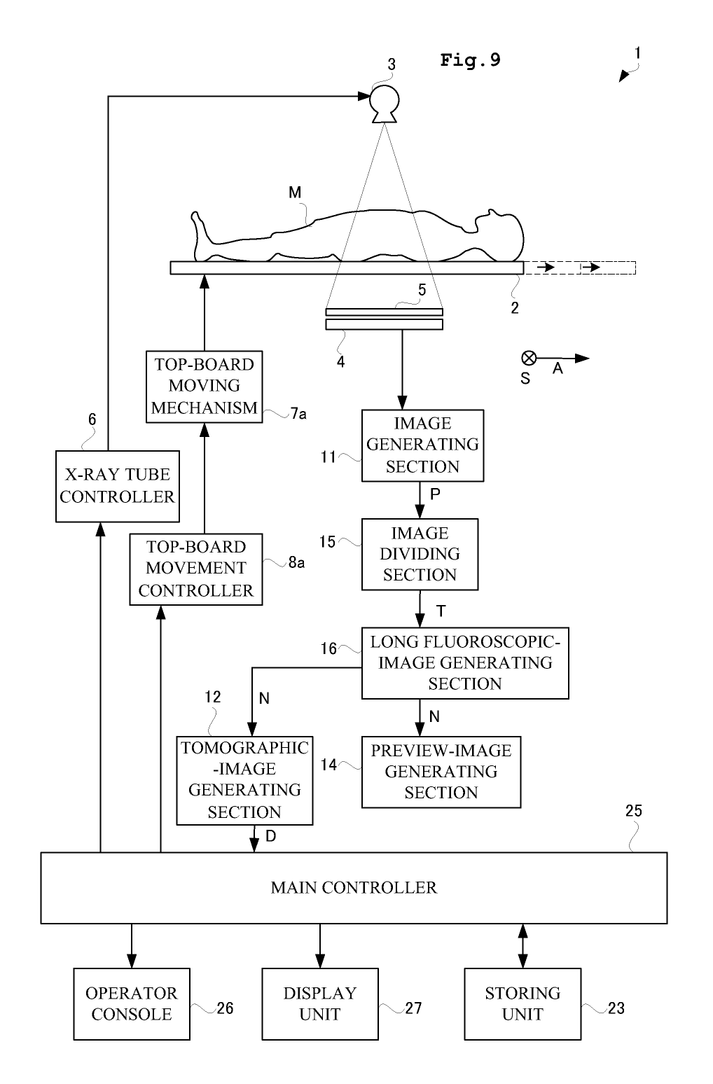

Description will be given next of a radiation tomography apparatus according to Example 2. FIG. 9 is a functional block diagram illustrating an X-ray apparatus according to Example 1 of this invention. As shown in FIG. 9, an X-ray apparatus 1 in Example 1 includes a top board 2 for supporting a subject M placed thereon as a target for X-ray tomography, an X-ray tube 3 disposed above the top board 2 (on one face side of the top board 2) for irradiating the subject with a cone-shaped X-ray beam, an FPD 4 below the top board 2 (on the other face side of the top board) for detecting X-rays transmitting through the subject M, a top-board moving mechanism 7a for moving the top board 2 relative to the X-ray tube 3 and the FPD 4 while a relationship between the X-ray tube 3 and the FPD 4 in which a center axis of the cone-shaped X-ray beam conforms to a center of the FPD 4 is maintained, a top-board movement controller 8a for controlling the top-board moving mechanism 7a, and an X-ray grid 5 provided as to cover an X-ray detecting surface of the FPD 4 that detects X-rays of the FPD 4 for absorbing scattered X-rays. As above, the top board 2 is placed between the X-ray tube 3 and the FPD 4. The X-ray tube 3 corresponds to the radiation source in this invention. The FPD 4 corresponds to the radiation-detecting device in this invention. Moreover, the top-board moving mechanism 7a corresponds to the moving device in this invention. The top-board movement controller 8a corresponds to the movement control device.

The X-ray tube 3 repeatedly irradiates the subject M with the cone-shaped and pulsed X-ray beam under control by the X-ray tube controller 6. The X-ray tube 3 has a collimator attached thereto for collimating the X-ray beam into a shape of a pyramid cone. The X-ray tube 3 and the FPD 4 constitute an imaging system for obtaining X-ray fluoroscopic images.

The X-ray apparatus 1 according to Example 1 further includes a main controller 25 for performing overall controlling en bloc each controller 6, 8 and a display unit 27 for displaying a tomographic image. The main controller 25 has a CPU, and provides each controller 6, 8 and each section 11, 12, 13, 14, 15, 16, to be mentioned later, by executing various programs. A storing unit 23 stores every parameter, such as a set value of a division width to which an image dividing section 15 refers upon operation thereof. The X-ray apparatus 1 refers to the parameter upon operation thereof.

The top-board moving mechanism 7a moves the top board 2 relative to the imaging system. The top-board moving mechanism 7a, under control of the top-board movement controller 8a, moves the top board 2 linearly along a linear track parallel to the body-axis direction A of the subject M (a longitudinal direction of the top board 2). Accordingly, the subject M placed on the top board 2 is moved relatively to the imaging system. A direction of movement of the top board 2 conforms to the longitudinal direction of the top board 2. The top-board moving mechanism 7a moves the top board 2 relative to the imaging system. Here, the body-axis direction A corresponds to the moving direction in this invention.

When the top-board moving mechanism 7a moves the top board 2 relative to the imaging system, a relative position of both 3 and 4 is maintained. That is, the cone-shaped X-ray beam having various X-ray focal points due to movement of X-ray tube 3 is always received on the entire X-ray detecting surface of the FPD 4. Thus, for one examination, 74 fluoroscopic X-ray images P, for example, are obtained while a positional relationship between the top board 2 and the imaging system is changed. Specifically, the top board 2 is moved from an initial position shown in solid lines through a position shown in dashed lines toward a position shown in chain lines illustrated in FIG. 9. The central axis of the cone-shaped X-ray beam always conforms to the center of the FPD 4 during radiography. The fluoroscopic X-ray image P corresponds to the fluoroscopic image in this invention.

An image generating section 11 is provided downstream of the FPD 4 for generating the fluoroscopic X-ray images P in accordance with detection signals outputted from the FPD 4 (see FIG. 9). Further provided downstream of the image generating section 11 are an image dividing section 15, a preview-image generating section 14, a long fluoroscopic-image generating section 16, and a tomographic-image generating section 12. The image dividing section 15 divides the fluoroscopic X-ray image P into strip segments T. The preview-image generating section 14 generates a preview image V. The long fluoroscopic-image generating section 16 combines the segments T while shifting to generate a long fluoroscopic image N. The tomographic-image generating section 12 generates a tomographic image D for diagnosis by reconstructing two or more long fluoroscopic images N.

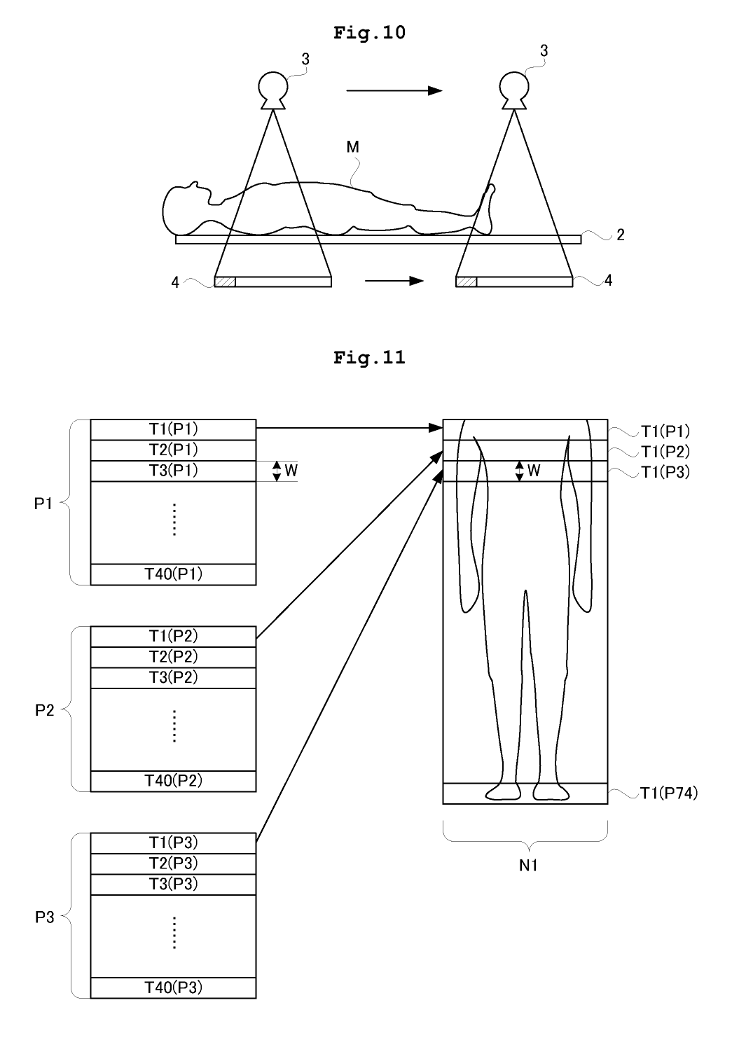

Next, description will be given of the principle of obtaining a tomographic image with the tomography X-ray apparatus 1 according to Example 1. FIG. 10 is a view illustrating a method of obtaining a tomographic image with the X-ray apparatus according to Example 2. FIG. 10 illustrates a positional relationship between the top board 2 and the imaging system when X-ray radioscopy is performed. In this example, radiography for fluoroscopic X-ray images P is started from the chest of the subject M and is completed at the tip of the foot of the subject M. It is supposed that 74 fluoroscopic X-ray images P are obtained from start until end of radiography, and each of the fluoroscopic X-ray images P having adjacent imaging times contains the image of the subject M shifting by a width W.

The image dividing section 15 divides the obtained fluoroscopic X-ray image P at equal intervals, and generates long strip segments T in the body-side direction S orthogonal to the body-axis direction A of the subject M (i.e. a direction of movement of the top board 2). That is, the image dividing section 15 divides each of the fluoroscopic X-ray images by a given width W in the direction of movement of the imaging system. The given width W is set by reading out a set value for determining the division width by the image dividing section 15 from the storing unit 23. Thus, a deviating width of the image of the subject M appearing in each of the fluoroscopic X-ray images P is equal to the width W of dividing the fluoroscopic X-ray image P. The set value read out by the image dividing section 15 is acquired in advance by geometrically calculating a width of movement of the subject image appearing on the FPD 4 as the imaging system are moved.

In comparison of the segments T generated by dividing the fluoroscopic X-ray image by the image division section 15, they differ from one another in incident direction of X-rays to the FPD 4. That is because X-rays emitted radially enter into the FPD 4 at different angles in accordance with positions of the FPD 4.

The image dividing section 15 divides all the obtained fluoroscopic X-ray images P as illustrated in FIG. 11. Since one fluoroscopic X-ray image P is divided into 40 pieces, 2,960 segments T are generated from 74 fluoroscopic X-ray images P through division. Moreover, the fluoroscopic X-ray images P are denoted by P1 to P74 in order from the upstream of the direction of movement of the imaging system. In addition, the segments generated by dividing a fluoroscopic X-ray image Pn into 40 pieces are denoted by T1(Pn) to T40(Pn) in order from the upstream of the direction of movement of the imaging system. That is, the segment T1(P1) contains a tomographic image of the subject M nearest to the head, whereas and the segment T40(P74) contains a tomographic image of the subject M nearest to the tip of the foot. Each of the segments T1(P1) to T40(P74) is sent to the long fluoroscopic-image generating section 16. The long fluoroscopic-image generating section 16 corresponds to the long fluoroscopic-image generating device in this invention.

The long fluoroscopic-image generating section 16 combines a segment T1(P1) of the fluoroscopic X-ray image P1 on the uppermost stream of the moving direction of the imaging system with each of the segments T1(P2) to T1(P74) of the fluoroscopic X-ray images P2 to P74 in the same position as the segment T1(P1) in order of radiography in the direction of movement of the imaging system. Specifically, as illustrated in FIG. 11, the segments T1(P1) to T1(P3) of the fluoroscopic X-ray images P1 to P3, respectively, in the upstream of the moving direction of the imaging system are combined in this order while sifting by the width W in the moving direction of the imaging system.

Then, a long-fluoroscopic image N1 can be obtained that extends in the body-axis direction A of the subject M. The long-fluoroscopic image N1 is obtained by shifting by the width W the segments T1(P1) to T1(P74) having an image of the subject M being contained therein while shifting the image by the width W in the body-axis direction A. Since the segment T has the width W in the body-axis direction A, when the segments T1(P1) to T1(P74) are arranged at a pitch of the width W to generate the long fluoroscopic image N1, the images of the subject M appearing in each of the segments can be combined sufficiently. Accordingly, the long-fluoroscopic X-ray image N1 contains the images of the subject M successively in the body-axis direction A, and thus no join between the segments can be confirmed.

Here, X-rays in the segments T1(P1) to T1(P74) constituting the long fluoroscopic image N1 enter in the same direction relative to the FPD 4. Consequently, the long fluoroscopic image N1 is an image when radiography is conducted to the subject M by parallel X-rays. The reason for the above is to be described. Firstly, each of the segments T1(P1) to T1(P74) is obtained at one end of the FPD 4 (see slashes of FIG. 10). That is, each of the segments constituting the long fluoroscopic image N1 is obtained within the same region of the FPD 4.

It is to be described how the incident direction of X-rays entering into this region varies during radiography. Specifically, since the relative position of the X-ray tube 3 and the FPD 4 does not vary during radiography, the incident direction of X-rays entering into one end of the FPD 4 (see slashes in FIG. 10) does not vary throughout radiography for a series of fluoroscopic X-ray images P. Accordingly, each of the segments T1(P1) to T1(P74) constituting the long fluoroscopic image N1 contains the image of the subject M when X-rays enter into the subject M in a given direction. Consequently, the incident direction of X-rays relative to the FPD 4 is uniform in the entire of the long fluoroscopic image N1.

The long fluoroscopic-image generating section 16 generates long fluoroscopic images N2 to N40 for other segments T2(P1) to T40(P1) generated from the fluoroscopic X-ray image P1 similarly to the segment T1(P1). The long fluoroscopic images N2 to N40 are similar to the long fluoroscopic image N1 in that the images of the subject M are contained successively in the body-axis direction A. Moreover, the long fluoroscopic images N2 to N40 are similar to the long fluoroscopic image N1 in that they are images when radiography is conducted to the subject M by parallel X-rays. However, the long fluoroscopic images N1 to N40 differ from one another in projection direction of the subject M. For instance, the long fluoroscopic image N1 is formed by the segments T1(P1) to T1(P74) that are obtained with X-rays being applied to the head of the subject M most obliquely, whereas the long fluoroscopic image N21 is formed by the segments T21(P1) to T21(P1) that are obtained with X-rays being applied to the subject M orthogonally.

As noted above, the long fluoroscopic-image generating section 16 combines a segment T, in a position of a fluoroscopic X-ray image, selected from the segments T generated by dividing the fluoroscopic X-ray image P and segments T in the same position as the segment T in each of the fluoroscopic X-ray images, thereby generating a long fluoroscopic image N. Then long fluoroscopic images N are successively generated for the other segments in a fluoroscopic X-ray image P (see FIG. 11).

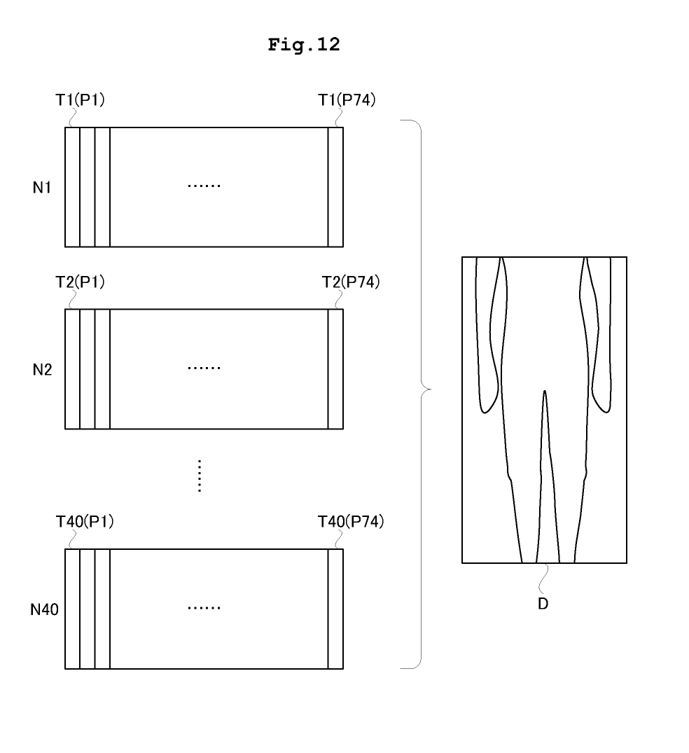

The long fluoroscopic images N1 to N40 are sent to the preview image generating section 14. The sent long fluoroscopic images N1 to N40 can be identified as an image when radiography is conducted to the subject M while the projection direction of the subject M varies. The structure within the subject M appears in each of the long-fluoroscopic images N1 to N40 while positions where the structure appears shift. The structure within the subject M has different shifting directions and speeds depending on whether the structure of the subject M lies near the X-ray tube 3 or near the FPD 4. The tomographic-image generating section 12 generates the tomographic image D for diagnosis when the subject M is cut at a cut position in the reference cut surface MA using various conditions of the structure appearing in each of the long fluoroscopic images N1 to N40 in accordance with positions in a level direction (i.e., a direction from the FPD 4 toward the X-ray tube 3). See FIG. 4. At this time, the preview-image generating section 14 generates the preview image V by filtered back projection (see FIG. 12).

A series of fluoroscopic images N1 to N40 are also sent to the tomographic-image generating section 12. The tomographic-image generating section 12 generates two or more tomographic images D for diagnosis at a cut position other than the reference cut surface MA by filtered back projection.

<Operation of X-Ray Apparatus>

Next, description will be given of operations of the X-ray apparatus 1. For conducting radiography to the subject M with use of an X-ray apparatus 1 according to Example 2, firstly a subject M is placed on the top board (which corresponds to Step S1 above). Thereafter, taking a fluoroscopic X-ray image P is started (which corresponds to Step S2 above.) Subsequently, a preview image V is generated based on a series of fluoroscopic X-ray images P taken (which corresponds to Step S3 above). The preview image generation section 14 generates the preview image V. Then a tomographic image D for diagnosis is generated based on a series of fluoroscopic X-ray images P (which corresponds to Step S4 above). Finally, the preview image V and the tomographic image D for diagnosis are displayed on a display unit 27 (which corresponds to Step S5 above).

Likewise in Example 2, the preview image V is displayed on the display unit 27 prior to the tomographic image D for diagnosis. Where the operator sees once the preview image V displayed on the display unit 27 and then determines that blur conditions of the subject M and exposure of X-rays are not suitable during radiography, parameters with respect to control of the X-ray tube 3 are adjusted via an operator console 26. Consequently, the process can return again to radiography of the fluoroscopic X-ray image P (Step S2.) Here, it is no need for the operator to wait for generation of the tomographic image D for diagnosis by the tomographic-image generating section 12.

This invention is not limited to the foregoing configurations, but may be modified as follows:

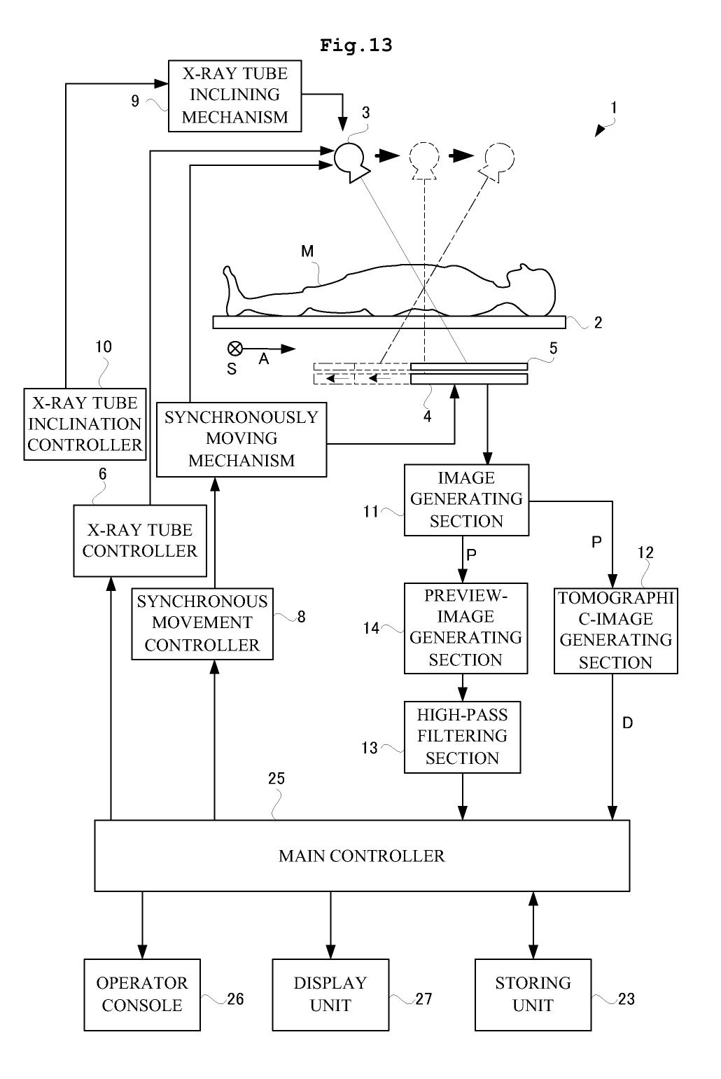

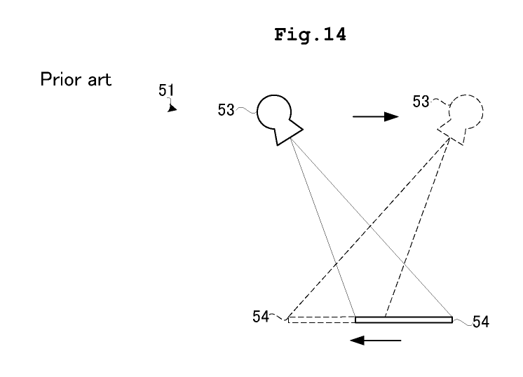

(1) In each example mentioned above, frequency process is performed on the fluoroscopic X-ray images and then they are superimposed to generate the preview image V. This invention is, however, limited to this. Specifically, as illustrated in FIG. 13, the high-pass filtering section 13 is provided downstream of the preview-image generating section 14. Fluoroscopic X-ray images having not been processed are superimposed to generate a preview image V. Thereafter, frequency process is performed for extracting components of the preview image V on a high-frequency side to display it on the display unit 27. Such configuration may be adopted. As noted above, the display unit 27 displays the preview image V having the components on the high-frequency side extracted therefrom, thereby displaying the preview image V with high visibility. In the method as in Example 1 of generating the preview image V by superimposing the fluoroscopic X-ray images P having the components on the high-frequency side extracted therefrom, image processing has to be performed to every fluoroscopic image. Accordingly, it takes time and effort for image processing. On the other hand, according to the configuration mentioned above, it is needed to perform image processing to the preview image V only. Consequently, the preview image V with high visibility can be provided through simpler calculations.

(2) In the examples mentioned above, the preview-image generating section 14 superimposes the fluoroscopic X-ray images P having the identical shape so as to cover them together completely, thereby generating the preview image V by superimposing a series of fluoroscopic X-ray images without shifting. This invention is, however, limited to this. Specifically, the preview-image generating section 14 superimposes the fluoroscopic X-ray images P while shifting by given intervals in the body-axis direction A of the subject M to generate the preview image V. Such configuration may be adopted. Accordingly, the preview image V having the sectional image in the cut surface other than the reference cut surface MA appearing therein can be generated.

(3) In the examples mentioned above, the device for executing the high-pass filtering section 13 and the preview image generating section 14 may differ from the device for executing the tomographic-image generating section 12. When the device for executing the high-pass filtering section 13 and the preview-image generating section 14 is replaced with a microcomputer, for example, operations of them are readily incorporable into the body of the X-ray apparatus 1.

(4) The examples mentioned above discuss a medical apparatus. This invention is applicable also to an apparatus for industrial use and for the nuclear field.

(5) X-rays used in the examples mentioned above are an example of radiation in this invention. Therefore, this invention may be adapted also for radiation other than X-rays.

1. A radiation tomography apparatus comprising:
a radiation source for emitting radiation to a subject;
a radiation detecting device for detecting the radiation emitted to the subject;
a top board for supporting the subject placed thereon between the radiation source and the radiation detecting device;
a moving device for moving the radiation source and the radiation detecting device in a direction of movement along the top board synchronously in opposite directions to each other;
a movement control device for controlling the moving device;
an image generating device for generating fluoroscopic images in accordance with detection signals outputted from the radiation detecting device;
a tomographic-image generating device for generating a tomographic image for diagnosis having a sectional image of the subject in any cut surface appearing therein by reconstructing a series of the fluoroscopic images successively taken while the radiation source and the radiation detecting device are moved;
a preview-image generating device for superimposing the series of the fluoroscopic images and for generating a preview image having the sectional image of the subject in a given cut surface appearing therein; and
a display device for displaying the preview image prior to displaying the tomographic image for diagnosis.

2. The radiation tomography apparatus according to claim 1, wherein
the preview-image generating device generates the preview image by superimposing the series of the fluoroscopic images having an identical shape without shifting.

3. The radiation tomography apparatus according to claim 1, wherein
the preview-image generating device generates the preview image by superimposing the series of the fluoroscopic images with addition-integration processing.

4. The radiation tomography apparatus according to claim 1, further comprising a high-pass filtering device for extracting components on a high-frequency side in the fluoroscopic image, wherein
the preview-image generating section generates the preview image by superimposing the fluoroscopic images having the components on the high-frequency side extracted therefrom by the high-pass filtering device.

5. The radiation tomography apparatus according to claim 1, further comprising a high-pass filtering device for extracting components on the high-frequency side in the preview image generated by the preview-image generating device, wherein
the display device displays the preview image having the components on the high-frequency side extracted therefrom.

6. A radiation tomography apparatus comprising:
a radiation source for emitting cone-shaped radiation to a subject;
a radiation detecting device for detecting the radiation emitted to the subject;
a top board for supporting the subject placed thereon between the radiation source and the radiation detecting device;
a moving device for moving an imaging system, constituted by the radiation source and the radiation detecting device, and the top board relatively to each other;
a movement control device for controlling the moving device;
an image generation device for generating fluoroscopic images in accordance with detection signals outputted from the radiation detecting device;
an image-dividing device for dividing, by a predetermined width in a direction of movement, each of the fluoroscopic images taken while the imaging system is moved relatively to the top board, and for generating segments having different incident directions of radiation relative to the radiation detecting device;
a long fluoroscopic-image generating device for combining the segments having an identical incident direction of radiation one another in the direction of movement in order of radiography, and for successively generating long fluoroscopic images for segments other than a segment in a fluoroscopic image;
a preview-image generating device for generating a preview image having the sectional image of the subject in a given cut surface appearing therein based on the long fluoroscopic images;
a tomographic-image generating device for generating a tomographic image by reconstructing two or more generated long fluoroscopic images; and
a display device for displaying the preview image prior to displaying the tomographic image for diagnosis.
